(19) United States
(12) Patent Application Publication
(10) Pub. No.: US 2010/0129300 A1
(43) Pub. Date: May 27, 2010

(54) ORGANOSILICON COMPOUNDS, FATTY ACIDS AND OILS WITH HOMOGENOUS SILICON NANOPARTICLE DISPERSIONS

(75) Inventors: Munir H. Nayfeh, Urbana, IL (US); Hanan I. Malkawi, Irbid (JO); Ebtesam Mohammed Salanh Al-Olayan, Riyadh (SA); Khalid Mohammad AlGhamdi, Riyadh (SA)

Correspondence Address: GREER, BURNS & CRAIN, 300 S WACKER DR, 25TH FLOOR, CHICAGO, IL, 60606, US

(73) Assignee: NANOSI ADVANCED TECHNOLOGIES, INC., Champaign, IL (US)

(21) Appl. No.: 12/620,080

(22) Filed: Nov. 17, 2009

Related U.S. Application Data
(60) Provisional application No. 61/115,772, filed on Nov. 18, 2008.

(51) Int. Cl. A61K 8/25; A61K 8/36; A61Q 1/02; A61Q 17/04

(52) U.S. Cl. 424/59; 424/63; 977/773

(57) ABSTRACT

The invention provides homogenous dispersions of luminescent silicon nanoparticles in organoslicoon compounds, fatty acids and oils. In methods of the invention, a fatty acid or oil and silicon nanoparticle homogenous dispersion is formed by mixing. The dispersion of nanoparticles in organosilicon compounds, fatty acid(s) or oil(s) can used as a delivery mechanism to homogenously incorporate the nanoparticles into a variety of cosmetic compounds and oils, such as foundation creams and other make-up products, sun tan lotions, sun tan oils, etc. Compounds of the invention with homogenous nanoparticle dispersions display homogeneous UV/blue absorption as well as down conversion to visible luminescence. The compounds can be tailored to exhibit white luminescence under UV illumination. Preferred skin care compounds provide a pleasing cosmetic effect in response to light. Preferred skin care compounds also provide uv protection.

### PRIORITY CLAIM AND REFERENCE TO RELATED APPLICATION

The application claims priority under 35 U.S.C.§119 from prior provisional application Ser. No. 61/115,772, which was filed on Nov. 18, 2008.

### FIELD

A field of the invention is organosilicon compounds, fatty acids and oils. Example applications of the invention include skin care products.

### BACKGROUND

There is a general interest in the incorporation of nanoparticle pigments into silicon substances, such as organosilicon compounds and silicone oils, as well as into non-silicone fatty substances, such as vegetable, animal, mineral or synthetic oils or fatty acid triglycerides Potential benefits that have been recognized include enhancing the performance and functionality of skincare products cosmetically.

Metal nanoparticles have been considered for their cosmetic properties, namely to serve as pigments. Proposed pigments have included precious metal nanoparticles (particularly, gold, silver, and platinum) and carbon fullereins as well as a variety of metal oxides including titanium dioxide (TiO₂), zinc oxide (ZnO), zirconium dioxide (ZrO₂), black, yellow, red and brown iron oxides, cerium dioxide (CeO₂) or alternatively the organic pigments known as barium, strontium, calcium and aluminum lakes. A few other oxides have also been proposed for cosmetic uses in skin care products, including cerium oxide (CeO), alumina (Al₂O₃), titanates (BaTiO₃, Ba_{0.5}Sr_{0.5}TiO₃, SrTiO₃), indium oxide (In₂O₃), tin oxide (SnO₂), antimony oxide (Sb₂O₃), magnesium oxide (MgO), calcium oxide (CaO), manganese oxides (Mn₃O₄, MnO₂), molybdenum oxide (MoO₃), silica (SiO₂), yttrium oxide (Y₂O₃), etc., and mixtures thereof.

Such metal oxide-based nanomaterials present synthesis challenges and safety issues. Most such materials lack solubility in cosmetically acceptable media. Control of colloids is difficult because thermodynamic and kinetic barriers inhibit the dispersal of inorganic, often hydrophilic (or hydrophobic) nanoparticles in hydrophobic (or hydrophilic) solvents without agglomeration caused by strong attractive ionic and van der Waals forces. Such materials can also adversely affect the applicability of skin care products, which must be amenable to easy application and provide easy and uniform spreading. Organic linkers that regulate the inter particle interaction as well as the particle-solvent interaction have been considered to address these applicability concerns, but such protocols add processing cost, and introduce unwanted chemicals that can themselves adversely affect the intended function. The additional chemicals can produce premature degradation and/or decreased thermo, mechanical, or optical properties of the compounds. There are also obvious safety and health concerns with toxic heavy metals and their oxides may also prove to be detrimental or at least undesirable because of the perceived potential for harm.

### SUMMARY OF THE INVENTION

The invention provides homogenous dispersions of luminescent silicon nanoparticles in organosilicon compounds, fatty acids and oils. In methods of the invention, a fatty acid or oil and silicon nanoparticle homogenous dispersion is formed by mixing. The dispersion of nanoparticles in organosilicon compounds, fatty acid(s) or oil(s) can used as a delivery mechanism to homogenously incorporate the nanoparticles into a variety of cosmetic compounds and oils, such as foundation creams and other make-up products, sun tan lotions, sun tan oils, etc. Compounds of the invention with homogenous nanoparticle dispersions display homogeneous UV/blue absorption as well as down conversion to visible luminescence. The compounds can be tailored to exhibit white luminescence under UV illumination. Preferred skin care compounds provide a pleasing cosmetic effect in response to light. Preferred skin care compounds also provide uv protection.

### BRIEF DESCRIPTION OF THE DRAWINGS

FIG. 1 shows the spectrum of an LED source and a 365 nm mercury lamp source taken with a holographic grating; the sources were used in experiments to test the luminescence of experimental compounds of the invention;

FIG. 2 shows the photoluminescence spectra changes for the silicon nanoparticle-fatty acid homogenous mixture for increasing concentration levels of silicon nanoparticles;

FIGS. 3A-3C show the chemical structures of some saturated and unsaturated fatty acids used in experiments; and

FIG. 4 shows the spectrum of nanoparticles in silicon oil along with a control spectrum of the silicon oil alone.

### DETAILED DESCRIPTION OF THE PREFERRED EMBODIMENTS

The invention provides homogenous dispersions of luminescent silicon nanoparticles in organosilicon compounds, fatty acids and oils. Certain preferred embodiments of the invention can be used directly as a cosmetic, such as when the oil is suitable as a skin oil. Additional embodiments of the invention use the organosilicon, acid and/or oil and silicon nanoparticle dispersion to deliver the silicon nanoparticles into another cosmetic product, such as foundation cream. In methods of the invention, an organosilicon compound, fatty acid or oil and silicon nanoparticle homogenous dispersion is formed by mixing. The silicon nanoparticles homogenously distribute to form a homogenous silicon nanoparticle organosilicon compound. The organosilicon compound, fatty acid or oil containing silicon nanoparticles can also be used as a delivery medium to deliver silicon nanoparticles into a cosmetic cream or oil, for example, foundation cream.

There is no significant agglomeration or separation of the particles from the compounds into which they are mixed. The mechanism for homogenous mixing and retention is believed to be formation a S—C linkage, resulting in homogeneous dissolution of particles with no agglomeration

In methods for synthesis of the invention, organosilicon compounds, fatty acid(s) or oil(s) are impregnated with ultra small silicon nanoparticles. The dispersion of nanoparticles in organosilicon compounds, fatty acid(s) or oil(s) can used as a delivery mechanism to homogenously incorporate the nanoparticles into a variety of cosmetic compounds and oils, such as foundation creams and other make-up products, sun tan lotions, sun tan oils, etc. Compounds of the invention with homogenous nanoparticle dispersions display homogeneous UV/blue absorption as well as down conversion to visible luminescence. The compounds can be tailored to exhibit white luminescence under UV illumination.

The homogeneous mixing mechanism is explained in terms of a saturating hydrosilylation process that incorporates the nanoparticles via formation of Si—C bonds at the double C═C bonds of the organosilicon compound(s), fatty acid(s), or oil(s). In an example embodiment, silicon nanoparticles are impregnated into highly volatile silicon oil, which is then used as a delivery substance to a cosmetic compound. Once delivered to a surface of cosmetic compound the silicon oil quickly evaporates leaving behind a homogeneous dispersion of silicon nanoparticles.

Preferred embodiments of the invention include skin care compounds with homogenous silicon nanoparticle distributions. Si nanoparticles add an attractive cosmetic appearance to skincare products in the form of offering attractive light effects. Additionally, the nanoparticle compounds can provide excellent UV protection, and one application of compounds of the invention is as a sun block product. Preferred embodiment cosmetic compounds can include many forms of make-up, creams, sun-blocks, oils, moisturizers, etc. The cosmetic compound mixes with an organosilicon compound, fatty acid or oil containing the homogenous dispersion of luminescent silicon nanoparticles. It is noted that the cosmetic compound can include metal nanoparticles, which should not interfere with the dispersion of silicon nanoparticles. Preferred embodiments of the invention will now be discussed with respect to the drawings. The drawings may include schematic representations, which will be understood by artisans in view of the general knowledge in the art and the description that follows. Features may be exaggerated in the drawings for emphasis, and features may not be to scale.

In preferred embodiments, silicon nanoparticles are mixed with fatty acid, an oil, or a silicon oil, such as organosilicon oil. Oils can also include standard cosmetic oils, sun tan oils, etc. In preferred embodiments, the silicon nanoparticles are formed on silicon wafer. In one embodiment, nanoparticles can be dispersed from the wafer directly into the oil or acid, such as by sonication. An ultra-sound treatment can separate silicon nanoparticles formed by methods such as in US. Pat. Nos. 6,585,947; 6,743,406; and 7,001,578 from a wafer and directly into an oil, fatty acid or organosilicon compound. In other embodiments, the silicon nanoparticles are delivered to the fatty acid, oil or organosilicon compound in alcohol or another solvent. The fatty acid, oil or organosilicon compound with silicon nanoparticles homogenously distributed can be used to deliver the nanoparticles to many forms of make-up, creams, sun-blocks, oils, moisturizers, etc. The cosmetic compound mixes with an organosilicon compound, fatty acid or oil containing the homogenous dispersion of luminescent silicon nanoparticles.

Experiments were conducted to produce homogeneous silicon nanoparticle dispersions in silicone or organosilicon substances or in non-silicon (hydrocarbon) acids and oils without surface modification of nanoparticles (as prepared). Experiments produced nano-oil composites that exhibit homogeneous white luminescence under 365 nm from a mercury lamp as well as under blue/UV LED lamp at 386-418 nm. The homogeneous color mixing is explained in terms of room temperature reduction process, in which the as prepared H-terminated particles act as a hydrosilylation reducer of the C═C double bonds in the unsaturated acid to form a S—C linkage, resulting in homogeneous dissolution of particles with no agglomeration.

The silicon nanoparticles in the experiments were dispersed from silicon wafers using chemical etching, but other known techniques for producing silicon nanoparticles can be used. Example techniques are disclosed in U.S. Pat. Nos. 6,585,947 & 6,743,406. The etching method results in silicon nanoparticles with multiple Si—H termination sites.

The fatty acid used in example experiments to produce homogenous fatty acid-silicon nanoparticle mixtures is a commercial brand of carthamus tinctorius (safflower) seed oil, with a small component of tocopheryl acetate (vitamin E). The mixture is a liquid at room temperature as it is rich in unsaturated fatty acids (oleic acid, linoliec acid). Unsaturated fatty acids are generally liquids at room temperature. Fatty acids rich in saturated acids (palmitic acid, stearic acid) are generally semisolids or solids at room temperature and are called fats. Greater degrees of unsaturation lower the melting point. Thus, generally, unsaturated fatty acids have lower melting points than their saturated counterparts. Additionally, the longer the chain the higher the melting point.

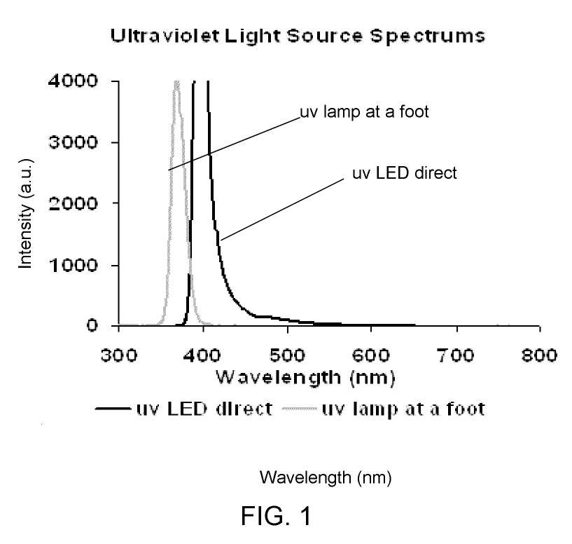

The photoluminescence of nanoparticle and acid mixtures was monitored under irradiation from a mercury lamp at 365 nm, and a blue/uv LED at 397 nm. The LED source consisted of 12 individual LEDs, configured in a similar housing to a regular household flash light. FIG. 1 shows the spectrum of the LED source as well as the 365 nm mercury source taken with a holographic grating. The LED band extends from 386 to 418 with a peak at 397 nm. The mercury band extends from 350 to 390 with a peak at 365 nm.

A control sample of fatty acid gives a weak blue/green luminescent band under the UV excitation. Red luminescent (−2.9 nm) silicon nanoparticles dissolved in isopropynol alcohol were added to a volume of the oil mixture in small increments and stirred vigorously. Other preferred embodiments include nanoparticles of different sizes, such as 1 nm (blue emitting), 1.67 (green emitting), 2.15 (yellow emitting), and 3.7 nm (infrared emitting) nanoparticles. Uniform dispersions of particles of a single size can be provided by the methods disclosed in U.S. Pat. No. 7,001,578, issued Feb. 21, 2006 and entitled Family of Discretely Sized Silicon Nanoparticles and Method for Producing the Same. In other embodiments, dispersions of silicon nanoparticles include nanoparticles of multiple sizes, and can be produced by a number of methods known in the art.

After each added increment of nanoparticles, using irradiation from the mercury lamp at 365 nm, and the blue/uv LED at 397 nm, the luminescence is found to be homogeneous as monitored by the unaided eye, as well as recorded by a holographic grating spectrum analyzer.

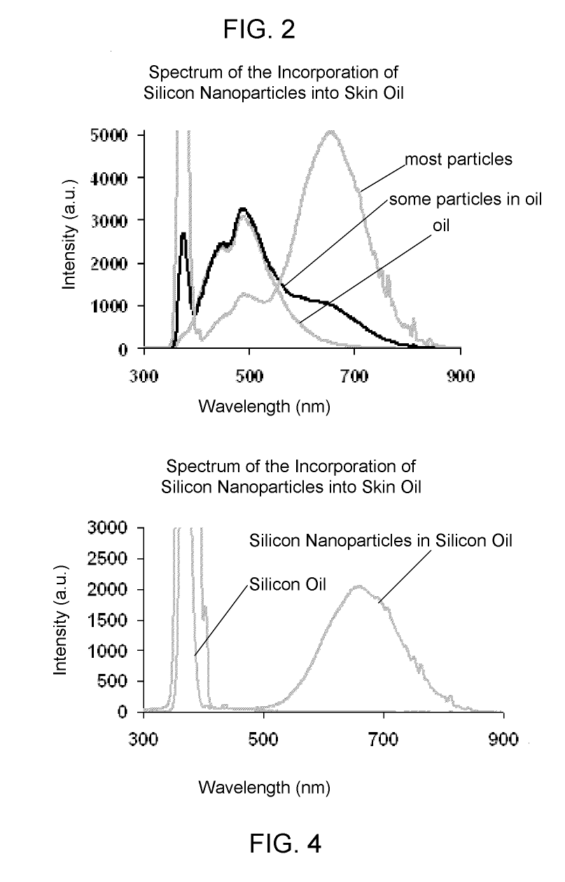

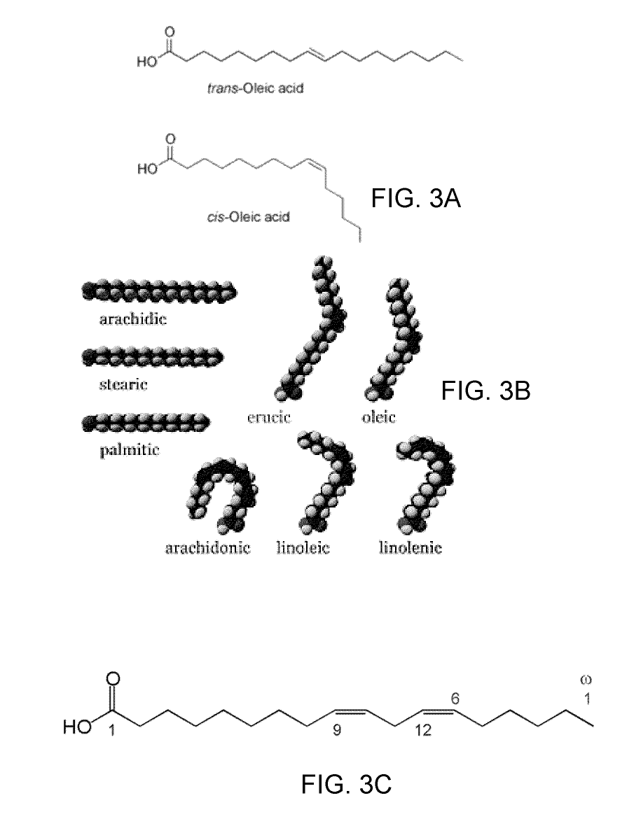

The gradual addition of nanoparticles showed how adding silicon nanoparticles changed the characteristic blue/green luminescence of the oil, to nearly white. Eventually, continued addition of silicon nanoparticles produces a red tinge, which is characteristic of that of the pure 2.9 nm silicon particle samples. FIG. 2 shows the photoluminescence spectra changes for the silicon nanoparticle-fatty acid homogenous mixture for increasing concentration levels of silicon nanoparticles. Pure oil shows a blue/green band extending from 427 nm to 607 nm with a peak at 515 nm and a tail extending to 670 nm. The pure silicon nanoparticles show a red band extending from 550 nm to 800 nm with a peak at 630 nm. The particle-oil homogenous mixture spectrum shows filled spectrum covering reasonably well most of the visible spectrum. The sample prepared in the experiments showed no local inhomogeneous formations.

The fatty acid-silicon nanoparticle dispersion can be used as a carrier to provide homogenous incorporation of silicon nanoparticles into a variety of cosmetic and skin care products. In example experiments, the dispersion of silicon nanoparticles in fatty acid was mixed into a commercial skin care foundation crème. The mixture showed homogeneous luminescence, indicating homogeneous dispersion. The dispersion can also be mixed, for example, into various skin lotions, creams, sun-blocks, etc.

The example fatty acids used for the experiments consisted of the three most abundant fatty acids in nature. These include saturated component: CH₃(CH₂)₁₄COOH palmitic acid (16:0), and CH₃(CH₂)₁₆COOH stearic acid (18:0), in addition to an unsaturated component: CH₃(CH₂)₇CH═CH(CH₂)₇COOH oleic acid (18:1), and CH₃(CH₂)₄CH═CHCH₂CH═CH(CH₂)₇COOH linoleic acid (18:2). Nearly all fatty acids consist of linear unbranched chains of long hydrophobic CH₂ section and a short hydrophilic carboxyl head, generally with an even number of carbon atoms, most between 12 and 20. In this notation, the number of carbon in the fatty acid and the number of unsaturated carbon-carbon double bonds in its hydrocarbon chain are shown by two numbers separated by a colon. Unlike saturated acids, which are in trans straight configuration, the oleic acid is in the cis configuration in which the chain is bent at the double bond site. In most unsaturated fatty acids, the cis isomer predominates; the trans isomer is rare. The lenoleic acid is also in the cis configuration with two bends, one at each of the double bonds. It is to be noted that fully saturating the double bonds in the oleic and the lenoleic acids straightens them and converts them to the stearic acid.

It is believed that the silicon nanoparticles may be incorporated in the process as a chemical phase at the carbon double bonds via hydrosilylation that saturates the acid. In this process, the Si atoms bond to C sites to produce silicon carbide bonds. Since the saturated version of the oleic acid is the stearic acid, this process effectively produce singly functionalized stearic acid with silicon nanoparticles. Similarly, the process in the case of the lenoleic acid could produce doubly functionalized stearic acid with nanoparticle. In this manner a small fraction of Si particles effectively dissolve in the oil uniformly and homogeneously without aggregation or agglomeration or disruption of the overall properties of the oil phase.

Another method of delivering the silicon nanoparticles to cosmetic creams used a volatile carrier. Using a volatile carrier, silicon nanoparticles can be delivered to the skin, a cosmetic cream, or various other cosmetic products. In experiments, a safe silicon-based organic cyclic siloxane, Octamethylcyclotetrasiloxane C₈H₂₄O₄Si₄, was used. The oil used came in a mixed form that consisted of greater than 90% octamethylcyclotetrasiloxane and a 1-5% capacity of decamethylcyclopentasiloxane. This form of cyclic siloxane is commonly used in personal care products. Octamethylcyclotetrasiloxane is an eight-membered silicone/oxygen ring with two methyl groups attached to each of the silicon atoms. A concentrated solution of silicon nanoparticles is mixed with the volatile silicon oil and in turn the nanoparticles seem to disperse homogeneously throughout the oil making a fluorescent solution of volatile silicon oil and silicon nanoparticles. Delivering the particles to cosmetics by use of the volatile silicon oil seems to be the most effective. Once the silicon nanoparticles are delivered to, i.e., the skin, the volatile silicon oil will evaporate leaving behind homogeneously dispersed silicon nanoparticles and any other substance that is desired. FIG. 4 shows the spectrum of nanoparticles immersed in silicon oil along with a control spectrum of the silicon oil alone.

It is not fully understood how the silicon nanoparticles dissolve into the volatile oil. One possibility is that the electronegative oxygen atoms in the ring of octamethylcyclotetrasiloxane attract the silicon nanoparticles through a dipole induced interaction. Dipole induced interaction occur when an electronegative atom plays the role of a dipole and induces a dipole in another atom or molecule. In octamethylcyclotetrasiloxane, oxygen plays the role of the dipole and in turn induces an attractive charge in the silicon nanoparticles and allows the silicon nanoparticles to thoroughly dissolve into the volatile silicon oil to create a solution of particles.

Applications

In addition to the pleasant cosmetic glow that can be provided by organosilicon compounds of the invention, there are skin care and protection applications that are of interest. UV protection is one application. The dispersions are also interesting from the point of view of UV protection. It is known that UV-B rays (280-320 nm) cause erythema and skin burns. It is also known that UV-A rays (320-400 nm), which cause tanning may induce damage, loss in elasticity, and the appearance of wrinkles, leading to premature aging especially in the case of sensitive skin Sun screen products on the market utilize multiple chemical ingredients to cover the entire UV range but mixtures tend to have poor photochemical stability, necessitating repeated applications at close and regular intervals. The trend now is to shift to other classes of filters. Metal (oxide) nanoparticle ingredients have been used as filters. However, heavy metal toxicity is an issue. The present nanoparticle organosilicon compounds provide protection against both UV-A and UV-B, while they are highly photostable and highly non-toxic. Because Si nanoparticles are highly absorbent to UV and have high quantum conversion efficiency, only a very small fraction is needed to achieve the required functionality.

Antioxidant effects might also be achieved with skin creams of the invention that consist of homogenous organosilicon silicon nanoparticle compounds. Vitamin E plays an antioxidant function in the body. Vitamin E is a linear chain of CH₃ with an aromatic ring head. It is an anti-oxidant agent that is known to capture hydroxyl HOO⁻. Oxidation of cells is believed to be the cause of aging. When cells are oxidized by peroxy radicals, such as HOO⁻ and ROO⁻, the properties of cell membranes change enough to cause the immune system to consider the cell an enemy, triggering spontaneous attack and destruction. Particles could alleviate this effect since radicals may be more inclined to extract electrons from the particles than from the cell membranes.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A skin care compound, comprising:
one of an organosilicon compound, fatty acid or oil or a combination thereof; and
a homogenous distribution of silicon nanoparticles throughout said one of an organosilicon compound, fatty acid or oil or combination thereof.

2. The skin care compound of claim 1, wherein said organosilicon compound, fatty acid or oil or a combination thereof is silicon oil.

3. The skin care compound of claim 1, wherein said organosilicon compound, fatty acid or oil or a combination thereof comprises a fatty silicon acid.

4. The skin care compound of claim 1, wherein said organosilicon compound, fatty acid or oil or a combination thereof comprises a fatty non-silicon acid

5. The skin care compound of claim 1, wherein said organosilicon compound, fatty acid or oil or a combination thereof comprises an unsaturated fatty acid.

6. The skin care compound of claim 1, wherein said organosilicon compound, fatty acid or oil or a combination thereof comprises a fatty acid with a saturated component and an unsaturated component.

7. The skin care compound of claim 6, wherein the saturated component comprises CH₃(CH₂)₁₄COOH palmitic acid and CH₃(CH₂)₁₆COOH stearic acid, and the unsaturated component comprises CH₃(CH₂)₇CH═CH(CH₂)₇COOH oleic acid, and CH₃(CH₂)₄CH═CHCH₂CH═CH(CH₂)₇COOH linoleic acid.

8. The skin care compound of claim 1, mixed into foundation skin cream.

9. The skin care compound of claim 1, wherein said organosilicon compound, fatty acid or oil or a combination thereof comprises carthamus tinctorius seed oil.

10. The skin care compound of claim 9, wherein said organosilicon compound, fatty acid or oil or a combination thereof further comprises tocopheryl acetate (vitamin E).

11. The skin care compound of claim 1, wherein said organosilicon compound, fatty acid or oil or a combination thereof comprises silicon-based organic cyclic siloxane.

12. The skin care compound of claim 1, mixed into one of a foundation cream, make-up product, sun tan lotion, skin lotion, and sun tan oil.

13. The skin care compound of claim 12, wherein said organosilicon compound, fatty acid or oil or a combination thereof further comprises tocopheryl acetate (vitamin E).

14. A method for preparing a silicon nanoparticle organosilicon compound, fatty acid or oil, the method comprising steps of:
providing silicon nanoparticles; and
mixing the silicon nanoparticles into one of an organosilicon compound, fatty acid or oil or combination thereof.

15. The method of claim 14, further comprising a step of delivering the silicon nanoparticle organosilicon compound, fatty acid or oil or combination thereof to a cosmetic compound.

16. The method of claim 15, wherein the cosmetic compound comprises one of foundation creams, make-up products, sun tan lotion, skin lotion and sun tan oil.

17. The method of claim 14, wherein said step of providing comprises providing the silicon nanoparticles in a solution, and said step of mixing comprises incrementally adding the solution while vigorously stirring.

18. The method of claim 15, wherein said step of providing comprises providing the silicon nanoparticles attached to a wafer, and said step of mixing comprises separating the nanoparticles from the surface while the wafer is immersed in the organosilicon compound, fatty acid or oil or combination thereof.
